(19) United States
(12) Patent Application Publication
(10) Pub. No.: US 2021/0139943 A1
(43) Pub. Date: May 13, 2021

(54) MEDIUM AND PROCESS FOR THE CULTURE AND SELECTIVE ISOLATION OF THE BACTERIUM ENTEROCOCCUS HIRAE

(71) Applicants: FONDATION MEDITERRANEE INFECTION, Marseille (FR); UNIVERSITE D'AIX MARSEILLE, Marseille Cedex 7 (FR); ASSISTANCE PUBLIQUE - HOPITAUX DE MARSEILLE, Marseille (FR); CENTRE NATIONAL DE LA RECHERCHE SCIENTIFIQUE (CNRS), Paris (FR); INSERM (INSTITUT NATIONAL DE LA SANTE ET DE LA RECHERCHE MEDICALE), Paris (FR)

(72) Inventors: Didier RAOULT, Marseille (FR); Saber KHELAIFIA, Marseille (FR); Marion BONNET, Marseille (FR)

(73) Assignees: FONDATION MEDITERRANEE INFECTION, Marseille (FR); UNIVERSITE D'AIX MARSEILLE, Marseille Cedex 7 (FR); ASSISTANCE PUBLIQUE - HOPITAUX DE MARSEILLE, Marseille (FR); CENTRE NATIONAL DE LA RECHERCHE SCIENTIFIQUE (CNRS), Paris (FR); INSERM (INSTITUT NATIONAL DE LA SANTE ET DE LA RECHERCHE MEDICALE), Paris (FR)

(21) Appl. No.: 16/621,366

(22) PCT Filed: May 30, 2018
(86) PCT No.: PCT/FR2018/051245

§ 371 Date: Dec. 11, 2019

(30) Foreign Application Priority Data
Jun. 13, 2017 (FR) 1755310

(51) Int. Cl. C12Q 1/04; C12N 1/20

(52) U.S. Cl. CPC C12Q 1/045; C12N 1/20

(57) ABSTRACT

The present invention relates to a specific culture medium for the culture and selective isolation of an Enterococcus hirae bacterium consisting of nutrients other than sugars from a basic culture medium for the culture of enterococci without aesculin, comprising inhibitors of Gram-negative and Gram-positive bacteria other than enterococci and preferably at least one antifungal compound characterized in that it comprises:
as inhibitor of Gram-positive bacteria other than enterococci, sodium chloride at a concentration of at least 20 g/L and not more than 60 g/L, and
as the only sugar, mannitol, and
as the only dye, an indicator dye that changes colour at a pH lower than the pH of said specific culture medium corresponding to the acidification of said specific is culture medium resulting from the consumption of mannitol.

The present invention relates to a novel medium and process for the culture and selective isolation of the commensal bacterium Enterococcus hirae in a biological sample, in particular stool.

Bacteria of the genus Enterococcus are Gram-positive cocci, found in the form of diplococci. These are commensal germs of the digestive tract, most often responsible for endocarditis and urinary tract infections [1]. The genus Enterococcus is classified in the domain Bacteria, phylum Firmicutes, class Bacilli, order Lactobacillales, family Enterococcaceae and finally branch Clostridium. At present, there are 58 different species of enterococci [1].

The most common enterococci found in clinical samples are Enterococcus faecalis, which represents 75 to 85% of the clinical strains of enterococci, and Enterococcus faecium, which represents 10 to 20% of the clinical strains of enterococci, with other species of the clinical strains of Enterococcus representing approximately 5%, namely Enterococcus hirae, E. casseliflavus, E. gallinarum and E. raffinosus [14].

Enterococcus hirae is a zoonotic pathogen, found in mammals and birds, that has rarely been isolated from human infection [2].

Currently, different solid culture media are used to select bacteria of the genus Enterococcus, such as bile aesculin azide (BEA) medium (Sigma-Aldrich, Saint Quentin Fallavier, France) [3], which allows the selective isolation of bacteria of the genus Streptococcus belonging to group D and bacteria of the genus Enterococcus. Media for isolating enterococci include mEI Agar (Difco, BD, Franklin Lakes, New Jersey, USA), Chromocult® enterococci (Merck, Darmstadt, Germany), and m-Enterococcus agar (Sigma-Aldrich) [4].

The objective of the present invention is to specifically select E. hirae from among other bacteria of the genus Enterococcus. Indeed, this E. hirae bacterium plays an important role in immunity, particularly in the treatment of breast cancer [5]. The presence of E. hirae in women with breast cancer has been shown to promote response to chemotherapy treatment. E. hirae is an “oncomicrobiotic”, it promotes the cancer fighting therapeutic effect of cyclophosphamide (CTX). Indeed, it activates anti-tumour immunity via the induction of Th17 cells and by increasing the ratio of cytotoxic cells to regulatory T cells [5].

The purpose of the present invention is to select E. hirae from biological samples, in particular from patient stools, in order to detect the presence or absence of this bacterium in the microbiota of these patients and thus predict their possible response to therapeutic treatment with cyclophosphamide [5].

The present invention consisted first of specifically selecting bacteria of the genus Enterococcus and more particularly E. faecalis, E. faecium and E. hirae on a culture medium and then differentiating E. hirae from other enterococci by a staining technique.

It is understood that the culture medium for Enterococcus according to the invention consists of a basic culture medium, known for the selective culture of enterococci, including different inhibitors of Gram-negative bacteria and most Gram-positive bacteria, other than enterococci.

A medium commonly used for the detection of enterococci is the bile-aesculin medium which includes:
10% beef bile (enterococci tolerate up to 40% bile unlike many other germs);
sodium azide (an antiseptic eliminates all Gram-negative. bacteria);
aesculin and ammoniacal iron citrate (a compound that turns the culture medium black when aesculin is hydrolysed).

Enterococci develop by hydrolysing aesculin so that as the enterococci grow the medium turns black: the blackening of the medium expresses the hydrolysis by ammoniacal iron citrate of aesculin to aesculetin which binds with iron.

An enterococci-specific solid culture medium, called m-Enterococcus agar medium [3] preferably comprises in the following amounts and weight proportions per 1 L:

Pancreatic digest of gelatin: 10 g (1%)

Yeast extract: 30 g (3%)

Sodium chloride: 15 g (1.5%)

Aesculin: 1 g (0.1%)

Sodium azide: 0.15 g (0.015%)

Cycloheximide: 0.05 g (0.005%)

Nalidixic acid: 0.25 g (0.025%)

Agar: 15 g (1.5%)

Sodium azide has an inhibitory action on Gram-negative bacteria and on all streptococci except those in group D.

Cycloheximide has an antifungal action.

Nalidixic acid is a quinolone antibiotic, used for its action on Gram-negative bacteria.

Sodium chloride inhibits Gram-positive bacteria other than Enterococcus, in particular the subgenus Streptococcus D can be inhibited by the salinity of a culture medium. Thus, Enterococcus can be selectively grown on a hypersaline medium.

Pancreatic digest of gelatin and yeast extract provide the necessary nutrients.

The present invention provides a specific culture medium for the culture and selective isolation of an Enterococcus hirae bacterium consisting of nutrients other than sugars from a base culture medium for the culture of enterococci without aesculin, comprising inhibitors of Gram-negative and Gram-positive bacteria other than enterococci and preferably at least one antifungal compound characterized in that it comprises:
as inhibitor of Gram-positive bacteria other than enterococci, sodium chloride at a concentration of at least 20 g/L and not more than 60 g/L, and
as the only sugar, mannitol, and
as the only dye, an indicator dye that changes colour at a pH lower than the pH of said specific culture medium corresponding to the acidification of said specific culture medium resulting from the consumption of mannitol.

In particular, the use of aesculin in the base medium is avoided because it stains all enterococci black as indicated above.

According to the present invention, it is taken advantage of the fact that E. hirae does not consume mannitol while mannitol is consumed by causing a decrease in the pH or acidification of said specific culture medium by all the other major Enterococcus bacteria likely to be present in clinical samples from human patients, particularly stools, namely E. faecalis, E. faecium, E. casseliflavus, E. gellinarum and E. raffinosus [14].

More specifically, said medium according to the invention is in solid form comprising a gelling agent preferably at a concentration of at least 1%, more preferably agar at a concentration of 1.5%. The medium according to the invention thus has the ability to isolate Enterococcus hirae by culture from a stool sample microbial flora composed of about 10¹⁰ bacteria/g stool including in practice about 400 different species. This environment exerts above all a selection action on the rest of the flora, thanks to the presence of inhibiting agents among other factors, making it possible to select among the 400 species three species only, Enterococcus hirae, Enterococcus faecalis and Enterococcus faecium, which are differentiated by the is combination of their mannitol fermentation properties generating a pH corresponding to that of an indicator dye, bromocresol purple.

Enterococcus faecalis and Enterococcus faecium together represent 95% of the enterococcal bacteria that can be found in clinical samples of human stool. To select these three species only, Enterococcus hirae, Enterococcus faecalis and Enterococcus faecium, Gram-positive other than enterococci sodium chloride is used at a concentration of at least 20 g/L and not more than 60 g/L, which does not affect the growth of Enterococcus hirae at these concentrations.

Bromocresol purple was chosen as an indicator dye not in relation to mannitol as such but because of its range of colour shift in relation to the pH corresponding in this case to that resulting from the consumption of mannitol by the species concerned in the medium of the invention.

Preferably, the medium according to the invention includes an inhibitor of enterococcal Gram-positive bacteria other than Enterococcus hirae, Enterococcus faecalis and Enterococcus faecium, in particular an inhibitor of Enterococcus durans, clindamycin, preferably at a concentration of at least 8 mg/L. This antibiotic eliminates this Enterococcus durans bacterium which, like Enterococcus hirae, does not ferment mannitol and appears to occur rarely in stool samples.

More particularly, the pH of said specific culture medium according to the invention is 7.3±0.2 and the indicator dye is bromocresol purple.

This indicator dye changes at a pH of 5.2 to 6.8, with a pH in this range of 5.2 to 6.8 corresponding to the acidification of a pH 7.3±0.2 culture medium; inoculated with at least one isolated colony of Enterococcus bacteria other than Enterococcus hirae.

More particularly, the specific culture medium according to the invention contains at least 10 g/L mannitol.

More particularly, said specific culture medium according to the invention includes nutrients other than mannitol in a concentration of not more than 20 g/L, preferably at least 10 g/L. This relatively high concentration of mannitol compared to other nutrients promotes the priority consumption of said sugar by Enterococcus other is than E. hirae on the one hand provides sufficient nutrients for the growth of E. hirae.

More particularly, said specific culture medium according to the invention includes bromocresol purple as indicator dye at a concentration of at least 25 mg/L.

More particularly, said specific culture medium according to the invention includes bromocresol purple as indicator dye at a concentration of at least 50 mg/L.

In a known way, these nutrients are energy sources and source of carbon, nitrogen or phosphorus.

More particularly, said specific culture medium according to the invention includes as nutrients other than sugars a basic culture medium for the culture of enterococci: vitamins, inorganic metal salts, in particular of metals such as Cu, Zn, Co, Ni, Bi, Ti, and nitrogen compounds.

More particularly, said specific culture medium according to the invention includes as source of vitamins, essential salts and nitrogen compounds:
a beef extract, and
proteose-peptone.

Peptone provides amino acids and peptides as a source of energy and carbon other than sugars.

More particularly, said specific culture medium according to the invention includes:
a beef extract at a concentration of 1 g/L, and
proteose-peptone at a concentration of 10 g/L.

More particularly, said specific culture medium according to the invention includes a gelling product preferably selected from agars and agar, preferably in a weight proportion of 0.5 to 5%, more preferably 1 to 2%.

More particularly, said specific culture medium according to the invention includes:
as Gram-negative bacteria inhibitors:
   sodium azide, and
   nalidixic acid at a concentration of not more than 100 mg/L, and
   colistin, and
as antifungal: cycloheximide.

More particularly, said specific culture medium according to the invention includes the following components, preferably in the following amounts and weight proportions per 1 L:

Proteose-peptone: 10 g (1%)

Beef extract: 1 g (0.1%)

Sodium chloride: 60 g (6%)

Mannitol 10 g (1%)

Sodium azide: 0.15 g (0.015%)

Cycloheximide: 0.05 g (0.005%)

Nalidixic acid: 0.10 g (0.010%)

Colistin 0.025 g (0.0025%)

Clindamycin 0.008 g (0.0008%)

Bromocresol purple: 0.05 g (0.005%)

Agar: 15 g (1.5%)

The present invention also provides a process for the selective culture and isolation of an Enterococcus hirae bacterium, characterized in that a biological sample containing or likely to contain an Enterococcus hirae bacterium and/or Enterococcus bacteria other than Enterococcus hirae is cultured at a temperature of 37° C. for a time sufficient to cause staining of Enterococcus bacteria other than Enterococcus hirae by said dye in a specific solid culture medium according to the invention.

More particularly, said specific culture medium according to the invention allows the selection of an Enterococcus hirae bacterium from a tested sample comprising other bacteria selected from E. faecalis, E. faecium, E. casseliflavus, E. gallinarum and E. raffinosus.

More particularly, the process according to the invention comprises the following steps wherein:

a) a dilution, preferably at least 3 successive 1/10 dilutions (i.e. a 10⁻³ dilution), is made from a stool sample at a rate of 0.10 to 0.50 g/mL in a buffer solution, preferably PBS buffer, and

b) a diluted stool sample, preferably 100 microlitres of diluted stool, is inoculated on said specific solid culture medium, and

c) after 72 hours of incubation, preferably at least 5 days of incubation, at 37° C., a said Enterococcus hirae bacterium is detected if a colony of non-discoloured bacteria is identified with respect to said bromocresol purple dye, and

d) preferably, it is confirmed that said colony of non-discoloured bacteria is of the species Enterococcus hirae by a MALDI-TOF mass spectrometric identification technique.

The identification of Enterococcus hirae by a MALDI-TOF mass spectrometric identification technique has been described [15].

According to the present invention, it has been discovered that after at least 72 hours, preferably 5 days of incubation on agars, a colouration can appear with a medium according to the invention for all enterococcus strains except for Enterococcus hirae, including yellowing of E. faecium colonies.

More particularly in step a) a series of several 1/10 dilutions of the initial sample are performed, preferably at least 5 (i.e. a 10⁻⁵ dilution) from a stool sample, particularly one taken with an inoculating loop, at a rate of 0.15 g/mL of PBS buffer, and in step b) a sample of each of the dilutions is inoculated on said specific solid culture medium according to the invention, preferably a sample of 100 microlitres of diluted stool is inoculated on said specific solid culture medium.

Preferably, said stool samples are first pre-incubated (before step a) at 37° C., preferably for at least 24 hours, in a specific liquid culture medium of the same composition as the specific solid culture medium but without agar and preferably without dye.

More particularly, a said pre-incubation is carried out with a said sample of 0.10 to 0.50 g/mL stool in a buffer solution, preferably 0.15 g/L in PBS, in 10 to 100 mL of is said specific liquid culture medium, preferably 40 mL respectively.

This pre-incubation makes it possible to increase the selective power of the culture medium according to the invention against E. durans, E. faecalis and E. faecium as reported in example B below and thus to observe a greater number of E. hirae colonies if necessary from the third 1/10 dilution (i.e. a 10⁻³ dilution) of the pre-incubated sample.

Other features and advantages of the present invention will appear in the light of the detailed description of the invention and the illustrative examples below.

### Example A

Since enterococci are resistant to high salt concentrations [1], in order to eliminate some Gram-positive bacteria other than enterococci and Gram-negative bacteria resistant to the inhibitors mentioned above, different concentrations of salt (sodium chloride) were tested to find the most appropriate one to add to the formulation of the present invention. Four concentrations were tested (60 g/L, 65 g/L, 70 g/L and 75 g/L). The results showed that the three strains of enterococci—E. hirae, E. faecium and E. faecalis—tested on these media had growth for each of the concentrations tested. However, after reading the agars at 24 hours, it was observed that the higher the salt concentrations, the smaller the colonies of E. hirae bacteria obtained.

To allow sufficient visibility of the colonies, the sodium chloride concentration selected for a selective medium according to the present invention is at least 20 g/L but not more than 60 g/L.

The enterococci most commonly represented in biological samples, particularly stool, are E. faecalis and E. faecium [2, 6, 7]. As the other enterococcal species are subdominant, no inhibitors of the other enterococci were added to the formulation of the present invention. However, some enterococci—Enterococcus casseliflavus, enterococcus gallinarum and Enterococcus raffinosus—are present in stool in quantities comparable to Enterococcus hirae [8]. However, the present invention makes it possible to differentiate them from Enterococcus hirae, as shown below.

The main objective of the present invention is to isolate E. hirae from E. faecium and E. faecalis. The latter two bacterial species being resistant to a large number of inhibitors to which E. hirae is resistant, it was not possible to eliminate them from the samples. The inventors then sought to differentiate them according to their metabolic profile. For this purpose, the inventors sought sugars consumed either exclusively by E. hirae or exclusively by E. faecium and E. faecalis. After reviewing the literature data, three sugars were selected: melibiose, raffinose and mannitol. Indeed, melibiose is consumed by E. hirae and in a variable way by E. faecium. Variable refers to the fact that consumption of this sugar by E. faecium is strain dependent. Raffinose and mannitol, in turn, are exclusively consumed by E. faecalis and E. faecium and not by E. hirae (see Table 1) [9, 10, 11].

To detect the consumption of sugar by bacteria, the inventors added to the medium of the present invention an indicator dye whose colour will vary according to the pH of the medium. Indeed, when the bacteria consume sugar, there is an acidification of the medium, which leads to a change in the indicator dye. To determine the most appropriate indicator dyer, the inventors tested several, including the following three: phenol red, bromocresol green and bromocresol purple. Based on concentrations in media already containing one of these indicator dyes, the inventors first chose to test a concentration of 25 mg/L [12] for each of the chosen indicators.

### 1) Tests of the Different Indicator Dyes

Phenol red is red in colour; it turns yellow when the pH is in the indicator's transition zone, which is between 6.6 and 8. The medium having a pH in the range of 7.3±0.2, the indicator started to turn yellow as soon as it was autoclaved, preventing its use in a medium according to the present invention.

Bromocresol green has a blue colour; it turns yellow when the pH is in the indicator's transition zone, which is between 3.8 and 5.4. This indicator dye requires an acidification of the medium that is too high to be able to visualize a real change in colour. Inventors thus observed a change towards green rather than yellow. The contrast between the E. faecalis and E. faecium colonies and the culture medium was therefore not great enough to differentiate them.

Bromocresol purple is purple in colour; it turns yellow when the pH is in the indicator's transition zone, which is between 5.2 and 6.8. The advantage of this indicator dye is that it does not require a strong lowering of pH to allow yellow colonies with a yellow halo underneath to appear. In addition, this transition zone is ideal since the pH of the present invention is 7.3±0.2. The inventors therefore chose this last indicator dye for the formulation of the present invention. Wishing to further improve contrast, the inventors increased the bromocresol purple concentration to 50 mg/L.

### 2) Determination of Sugar

Once the indicator dye was chosen, the inventors were able to determine which sugar was most suitable for the present invention.

Melibiose gave variable results, due to the variable consumption of this sugar by E. faecium. This sugar cannot therefore be used to isolate E. hirae.

Concerning raffinose, the test was inconclusive because no colour change of the E. faecalis and E. faecium colonies could be observed.

Finally, the last sugar tested was mannitol. For this sugar, the inventors found that there was indeed a change in the colour of the medium and the colonies for E. faecalis and E. faecium, but not for those of E hirae which remained colourless. The inventors therefore chose mannitol as sugar for the formulation of said invention, at a concentration of 10 g/L, a concentration frequently found in culture media [12].

TABLE 1
Sugar consumption by the three enterococci studied

| | Melibiose | Raffinose | Mannitol |
|---|---|---|---|
| Enterococcus hirae | + | + | − |
| Enterococcus faecalis | − | − | + |
| Enterococcus faecium | V | − | + |

In addition, it has been shown in the literature that some strains of enterococci—Enterococcus casseliflavus, enterococcus gallinarum and Enterococcus raffinosus—are present in quantities comparable to E. hirae. Since bacteria belonging to the genus Enterococcus are generally sensitive and resistant to the same inhibitors, the inventors investigated the consumption of sugars for these different enterococci. It was found that they all consumed mannitol (see Table 2). It is therefore possible to distinguish E. hirae among these enterococci.

TABLE 2
Consumption of mannitol by enterococcal species present
in a quantity comparable to E. hirae in stool.

| | Mannitol |
|---|---|
| Enterococcus hirae | − |
| Enterococcus casseliflavus | + |
| Enterococcus gallinarum | + |
| Enterococcus raffinosus | + |

Said culture medium also includes a source of vitamins, essential salts and nitrogen compounds by the presence of beef extract [13] at a concentration of 1 g/L and protein peptone added at 10 g/L to said invention which provides amino acids and peptides (source of energy and carbon). No other nutrients have been added to the present invention because the objective is that E. faecalis and E. faecium should as a priority use sugar, mannitol, as source of nutrition for their growth. However, these sources cannot be removed since E. hirae, not using the selected sugar as an energy source, needs nutrients to grow.

Said culture medium is a solid culture medium containing a gelling agent preferably selected from agars, preferably in a weight proportion of 0.5 to 5%, more preferably 1 to 2%.

A culture medium for selecting Enterococcus hirae according to the invention includes the following components, preferably in the following amounts and weight proportions per 1 L of water.

### Medium A:

Proteose-peptone: 10 g (1%)

Beef extract: 1 g (0.1%)

Sodium chloride: 60 g (6%)

Mannitol 10 g (1%)

Sodium azide: 0.15 g (0.015%)

Cycloheximide: 0.05 g (0.005%)

Nalidixic acid: 0.25 g (0.025%)

Bromocresol purple: 0.05 g (0.005%)

Agar: 15 g (1.5%)

All the compounds in this medium are available from Sigma-Aldrich.

More particularly, a sample containing an Enterococcus hirae bacterium is cultured at a temperature of 37° C. for 72 hours in said Enterococcus hirae selection culture medium.

More particularly, the following steps are carried out:
a biological sample that may contain Enterococcus hirae is cultured. First, a series of six 1/10 dilutions is carried out from a stock solution of the initial sample. One hundred microlitres of each of the dilutions are inoculated on agar corresponding to the present invention, and
after 72 hours of incubation at 37° C., the uncoloured bacterium detected is identified as a bacterium of the species Enterococcus hirae by a MALDI-TOF mass spectrometry technique [15]. An Enterococcus hirae culture medium according to the invention allows E. hirae to be selected after 72 hours.

Examples 1 to 3 below provide results of the inoculation of a series of six dilutions of various samples containing three enterococci—E. hirae, E. faecalis and E. faecium—on a solid culture medium according to the present invention after 72 hours of incubation at 37° C.

Procedure:

In the three examples, a series of six dilutions from 10⁻¹ to 10⁻⁶ of a starting sample is performed as follows:
in example 1, a colony of a strain of Enterococcus hirae, of a strain of Enterococcus faecalis and of a strain of Enterococcus faecium were mixed in one millilitre of phosphate-buffered saline (PBS),
in example 2, a stool is artificially enriched with E. hirae, a colony of a strain of Enterococcus hirae was mixed in one millilitre of PBS with the equivalent of a blue inoculating loop (10 microlitres) of stool, i.e. about 0.15 g, and
in example 3, the equivalent of a blue inoculating loop (10 microlitres) of clinical stool, i.e. about 0.15 g known to be rich in E. hirae, was mixed in one millilitre of PBS.

To achieve these dilutions, the inventors prepared six Eppendorf tubes (Sigma-Aldrich), which were filled with 900 microlitres of PBS and, from the stock tube, containing the colonies and if necessary stool samples, 100 microlitres were collected and mixed with the second tube containing the 900 microlitres of PBS, and cascade dilutions were performed until six dilutions from 10⁻¹ to 10⁻⁶ were obtained.

Then 100 μL is inoculated on a solid culture medium according to example A above, from the series of six dilutions and the result is observed after the agars have been incubated in an oven at 37° C. for 72 hours.

For the three examples, yellow colonies appear for E. faecium and E. faecalis and transparent colonies for E. hirae. Isolated colonies are most visible on the 10⁻⁵ and 10⁻⁶ dilutions. The other dilutions are too concentrated in bacteria for isolated colonies to be observed and thus distinguish Enterococcus hirae from other enterococci for the analysis of the Enterococcus hirae richness of the tested stool.

Example B: By testing a larger number of samples, namely more than 100 clinical stools from the bacteriology diagnostic laboratory, it appeared that the medium of example A above, selected in rare occurrences (4 samples out of 100 tested) the Enterococcus durans bacterium together with Enterococcus hirae, both not fermenting mannitol and appearing transparent despite the BCP indicator dyer unlike the only two other species present, E. faecium and E. faecalis.

To overcome this, and to be even more selective for Enterococcus hirae, an antibiotic was found that inhibits the growth of E. durans, namely clindamycin, an antibiotic of the lincosamide family. This antibiotic has a mainly bacteriostatic action against aerobic Gram-positive bacteria including Enterococcus durans but apart from the three species E. faecium, E. faecalis and E. hirae and against a wide spectrum of is anaerobic bacteria. This antibiotic was added to the medium at a concentration of 8 mg/L.

In addition, the concentration of nalidixic acid was reduced from 250 mg/L to no more than 100 mg/L due to its poor dilution in water above 100 mg/L and colistin, a polypeptide antibiotic of the polymyxin family with action on Gram-negative bacteria, was added at a concentration of 25 mg/L to compensate.

By virtue of this medium B, it was possible to specifically select the following strains: Enterococcus hirae, Enterococcus faecalis and Enterococcus faecium.

For this purpose, use was made of a selective medium B comprising bacterial inhibitors such as 60 g/L NaCl, 0.05 g/L cycloheximide, 0.15 g/L sodium azide, 0.1 g/L nalidixic acid, 0.025 g/L colistin and 0.008 g/L clindamycin.

The formula for medium B is therefore:

### Proteose-peptone: 10 g (1%)

Beef extract: 1 g (0.1%)
Sodium chloride: 60 g (6%)

### Mannitol 10 g (1%)

Sodium azide: 0.15 g (0.015%)

### Cycloheximide: 0.05 g (0.005%)

Nalidixic acid: 0.10 g (0.010%)

### Colistin 0.025 g (0.0025%)

### Clindamycin 0.008 g (0.0008%)

Bromocresol purple: 0.05 g (0.005%)

### Agar: 15 g (1.5%)

To test the effectiveness of this medium, the inventors inoculated 100 random stools from the IHU Méditerranée Infection diagnostic laboratory (bacteriology). For this purpose, the equivalent of a blue inoculating loop (10 microlitres) of clinical stool, i.e. approximately 0.15 g, was mixed in 1 mL of PBS. The inventors decided to inoculate the 10⁻³ dilution on their medium, so as to observe a large number of colonies on the medium, in case of bacterial growth. To make dilutions, the inventors prepared is three Eppendorf tubes (Sigma-Aldrich), which were filled with 900 microlitres of PBS, and from the stock tube, containing the stool samples in 1 mL, 100 microlitres were taken and mixed with the second tube containing the 900 microlitres of PBS, and cascade dilutions were made until three dilutions from 10⁻¹ to 10⁻³ were obtained.

Then, 50 μL was inoculated on a solid culture medium according to example—B above, of the 10⁻³ dilution and the results are observed after the agars were incubated in an oven at 37° C. for 72 hours, preferably up to 5 days.

Of the 100 stools inoculated, only 40 showed bacterial growth. After identifying the colonies with the MALDI TOF SP spectrometer, the inventors observed that the only bacteria obtained were Enterococcus hirae (2), E. faecium (16), E. faecalis (22) and E. durans (4).

To eliminate E. durans, the inventors tested an antibiotic, clindamycin. This choice was based on a series of antibiotic susceptibility tests that the inventors had previously performed. In order to determine the best concentration of clindamycin to add to the culture medium, the inventors tested three concentrations of this antibiotic, namely 2 mg/L, 4 mg/L and 8 mg/L. The inventors inoculated strains of Enterococcus hirae, E. faecium, E. faecalis and E. durans. Growth for each of these species was observed for the first two concentrations of clindamycin, 2 mg/L and 4 mg/L. On the other hand, a lack of growth for E. durans was found at a concentration of 8 mg/L clindamycin. Enterococcus hirae, E. faecium, E. faecalis have growth at this concentration of clindamycin.

In order to confirm these results, the inventors retested the 4 stools where E. durans had been found. For each of these stools, no colony of E. durans was identified by MALDI TOF SP mass spectrometry. To increase the selective power of the culture medium against E. durans in the samples tested, the inventors pre-incubated the samples for 24 hours in empty anaerobic blood culture bottles and added the hirae culture medium, in its liquid form, mentioned below, at a rate of 40 mL per bottle. For this purpose, the equivalent of a blue inoculating loop (10 microlitres) of clinical stool, is containing E. durans, i.e. approximately 0.15 g, was mixed in 1 mL of PBS, and the whole was injected into the blood culture bottle. The inventors incubated the bottle at 37° C. for 24 hours.

They then made dilutions, as described above, and inoculated the third dilution (10⁻³) on the solid hirae medium. After 72 hours of incubation, preferably 5 days, no growth of E. durans could be observed.

### Liquid Hirae Culture Medium:

Proteose-peptone: 10 g (1%)

Beef extract: 1 g (0.1%)

Sodium chloride: 60 g (6%)

Mannitol 10 g (1%)

Sodium azide: 0.15 g (0.015%)

Cycloheximide: 0.05 g (0.005%)

Nalidixic acid: 0.10 g (0.010%)

Colistin 0.025 g (0.0025%)

Clindamycin 0.008 g (0.0008%)

The agar was removed to obtain a liquid medium, and the indicator dye, bromocresol purple, was also removed from this formula of the liquid medium because it was not useful during pre-incubation.

In addition, to promote the growth of E. hirae on the hirae culture medium, particularly when E. faecium and E. faecalis are also present in the tested stool sample, a pre-incubation of these samples is also performed for 24 hours. For this purpose, the equivalent of a blue inoculating loop (10 microlitres) of clinical stool, containing E. hirae, namely about 0.15 g, was mixed in 1 mL of PBS and injected into the blood culture bottle containing 40 mL of liquid hirae medium. The inventors incubated the bottle at 37° C. for 24 hours. They then made dilutions, as described above, and inoculated the third dilution (10⁻³) on the solid hirae medium. After 72 hours of incubation, preferably 5 days, a higher growth of E. hirae could be observed. Without pre-incubation, the inventors could observe fewer than 10 E. hirae colonies and after pre-incubation, this growth was of the order of 200-300 colonies at the same dilution.

### REFERENCES

1. Aguilar-Galvez A, Dubois-Dauphin R, Destain J, Campos D, Thonart P. Les entérocoques: avantages et inconvénients en biotechnologie (synthèse bibliographique). Biotechnologie. Agronomie. Société. Environnement. 2012 16(1), 67-76

2. Bourafa N, Loucif L, Boutefnouchet N and Rolain J -M. Enterococcus hirae, an unusual pathogen in humans causing urinary tract infection in a patient with benign prostatic hyperplasia: first case report in Algeria. New Microbe and New Infect. 2015; 8: 7-9

3. Rodier J, Legube B, Merlet N, Brunet R. L'analyse de l'eau—10e éd.: Eaux naturelles, eaux résiduaires, eau de mer. In: Dunod, editor. 2016 p 611

4. Maheux A, Bouchard S, Bérubé E and Bergeron M. Rapid molecular identification of fecal origin-colonies growing on Enterococcus spp.-specific culture methods. Journal of water and health. 2017; doi: 10.2166/wh.2016.199

5. Daillère R et al. Enterococcus hirae and Barnesiella intestinihominis Facilitate Cyclophosphamide-Induced Therapeutic Immunomodulatory Effects. Immunity. 2016; 45: 931-943

6. Savini V, Bonfini T, Marrollo R, Argentieri A V, Riccioni S, Astolfi D, Fazii P, D'Antonio D, Gherardi G. Enterococcus hirae: a zoonotic microorganism in human umbilical cord blood. World Journal of Microbiology and Biotechnology. April 2014; 30(4):1423-6

7. Zhanel G, Hoban D and Karlowsky J. Nitrofurantoin Is Active against Vancomycin-Resistant Enterococci. Antimicrobial Agents and Chemotherapy. January 2001; 45(1): 324-326.

8. Pinto B, Pierotti R, Canale G, Reali D. Characterization of ‘faecal streptococci’ as indicators of faecal pollution and distribution in the environment. Letters in Applied Microbiology. October 1999; 29(4):258-63

9. Berger S. GIDEON Guide to Medically Important Bacteria: 2017 edition. In: GIDEON Informatics Inc, editor. 2017 p 761-782

10. Devriese L, Van De Kerckhove A, Kilpper-Bälz R, Schleifer K. Characterization and Identification of Enterococcus Species Isolated from the Intestines of Animals. International Journal of Systematic and Evolutionary Microbiology. July 1987; 37: 257-259

11. Schleifer K, Kilpper-Bälz R. Transfer of Streptococcus faecalis and Streptococcus faecium to the Genus Enterococcus nom. rev. as Enterococcus faecalis comb. nov. and Enterococcus faecium comb. nov. International Journal of Systematic and Evolutionary Microbiology. January 1984; 34: 31-34

12. Microbiological culture media [Internet]. Available at: http:/www2.ac-lyon.fr/enseigne/biotech/microbio/milieux.html

13. Beef extract [Internet]. Available at: http://www.mastgrp.com/IFUS/IFU606_FR.pdf

14. Leclercq R. Faut-il identifier les entérocoques, et comment? La Lettre de l'Infectiologue—Tome XVI—n° 7—septembre 2001

15. Seng P, Abat C, Rolain J M, Colson P, Lagier J -C, Gouriet F, Pierre Edouard Fournier, Michel Drancourt, Bernard La Scola and Didier Raoult. Identification of rare pathogenic bacteria in a clinical microbiology laboratory: Impact of Matrix-Assisted Laser Desorption Ionization—Time of Flight Mass Spectrometry. J Clin Microbiol. 2013; 51(7):2182 94.

1. Specific culture medium for the culture and selective isolation of an Enterococcus hirae bacterium consisting of nutrients other than sugars from a base culture medium for the culture of enterococci without aesculin, comprising inhibitors of Gram-negative and Gram-positive bacteria other than enterococci and preferably at least one antifungal compound characterized in that it comprises:
as inhibitor of Gram-positive bacteria other than enterococci, sodium chloride at a concentration of at least 20 g/L and not more than 60 g/L, and
as the only sugar, mannitol, and
as the only dye, an indicator dye that changes colour at a pH lower than the pH of said specific culture medium corresponding to the acidification of said specific culture medium resulting from the consumption of mannitol.

2. Specific culture medium according to claim 1, characterized in that it comprises an inhibitor of enterococcal Gram-positive bacteria other than Enterococcus hirae, Enterococcus faecalis and Enterococcus faecium, in particular Enterococcus durans inhibitor, clindamycin, preferably at a concentration of at least 8 mg/L clindamycin.

3. Specific culture medium according to claim 2 characterized in that the pH of said specific culture medium is 7.3±0.2 and the indicator dye is bromocresol purple.

4. Specific culture medium according to claim 1, characterized in that it comprises at least 10 g/L of mannitol.

5. Specific culture medium according to claim 1, characterized in that it comprises nutrients other than mannitol in a concentration of not more than 20 g/L, preferably at least 10 g/L.

6. Specific culture medium according to claim 1, characterized in that it includes bromocresol purple as indicator dye at a concentration of at least 25 mg/L.

7. Specific culture medium according to claim 1, characterized in that it includes bromocresol purple as indicator dye at a concentration of at least 50 mg/L.

8. Specific culture medium according to claim 1, characterized in that it comprises as nutrients other than sugars of a basic culture medium for the culture of enterococci: vitamins, inorganic metal salts and nitrogen compounds.

9. Specific culture medium according to claim 8 characterized in that it comprises as source of vitamins, essential salts and nitrogen compounds:
a beef extract, and
proteose-peptone.

10. Specific culture medium according to claim 9 characterized in that it comprises:
a beef extract at a concentration of 1 g/L, and
proteose-peptone at a concentration of 10 g/L.

11. Specific culture medium according to claim 1, characterized in that it comprises a gelling product preferably selected from agars, preferably in a weight proportion of 0.5 to 5%, more preferably 1 to 2%.

12. Specific culture medium according to claim 1 characterized in that it comprises:
as Gram-negative bacteria inhibitors:
sodium azide, and
nalidixic acid at a concentration of not more than 100 mg/L, and
colistin, and
as antifungal: cycloheximide.

13. Specific culture medium according to claim 1 characterized in that it is in solid form comprising a gelling agent preferably at a concentration of at least 1%, more preferably agar at a concentration of 1.5%.

14. Specific culture medium according to claim 1, characterized in that it comprises the following components, preferably in the following amounts and weight proportions per 1 L:
Proteose-peptone: 10 g (1%)
Beef extract: 1 g (0.1%)
Sodium chloride: 60 g (6%)
Clindamycin: 0.008 g (0.0008%)
Sodium azide: 0.15 g (0.015%)
Cycloheximide: 0.05 g (0.005%)
Nalidixic acid: 0.10 g (0.025%)
Colistin: 0.025 g (0.0025%)
Mannitol 10 g (1%)
Bromocresol purple: 0.05 g (0.005%)
Agar: 15 g (1.5%)

15. Process for the selective culture and isolation of an Enterococcus hirae bacterium characterized in that a biological sample containing or likely to contain an Enterococcus hirae bacterium and/or Enterococcus bacteria other than Enterococcus hirae is cultured at a temperature of 37° C. for a time sufficient to produce a staining of Enterococcus bacteria other than Enterococcus hirae by said dye in a said specific solid culture medium according to claim 1.

16. Culture process according to claim 15 characterized in that an Enterococcus hirae bacterium is selected from a tested sample comprising other bacteria selected from E. faecalis, E. faecium, E. durans, E. casseliflavus, E. gallinarum and E. raffinosus.

17. Process according to one of claim 15 characterized in that the following steps are carried out:
a) a dilution, preferably at least 3 successive 1/10 dilutions (i.e. a 10⁻³ dilution), is made from a stool sample at a rate of 0.10 to 0.50 g/mL in a buffer solution, preferably PBS buffer, and
b) a diluted stool sample, preferably a sample of 100 microlitres of diluted stool, is inoculated on said specific solid culture medium according to the invention, and
c) after 72 hours, preferably at least 5 days of incubation, at 37° C., a said Enterococcus hirae bacterium is detected if a colony of non-discoloured bacteria is identified with respect to said bromocresol purple dye, and
d) preferably, it is confirmed that said colony of non-discoloured bacteria is of the species Enterococcus hirae by a MALDI-TOF mass spectrometric identification technique.

18. Culture process according to claim 15, characterized in that said stool samples are first pre-incubated at 37° C., preferably for at least 24 hours, in a specific liquid culture medium of the same composition as said specific solid culture medium but without agar and preferably without dye.

19. Culture process according to claim 18 characterized in that said pre-incubation is first carried out with a said sample of 0.10 to 0.50 g/mL stool in a buffer solution, preferably PBS, in 10 to 100 mL of said specific liquid culture medium.
